(19) United States
(12) Patent Application Publication
(10) Pub. No.: US 2002/0018777 A1
(43) Pub. Date: Feb. 14, 2002

(54) STERILIZATION OF VIRUS INFECTED PLASMA

(76) Inventors: ALFRED M. PRINCE, POUND RIDGE, NY (US); BERNARD HOROWITZ, NEW ROCHELLE, NY (US)

Correspondence Address: CRAIG J. ARNOLD, ESQ., AMSTER, ROTHSTEIN & EBENSTEIN, 90 PARK AVENUE, NEW YORK, NY, 10016

(21) Appl. No.: 09/204,703

(22) Filed: Dec. 3, 1998

Related U.S. Application Data
(63) Continuation of application No. 08/369,045, filed on Jan. 5, 1995, now abandoned.

(51) Int. Cl. A61K 39/395; C07K 16/00; A61K 39/40; A61K 39/42; C07K 14/00; C07K 17/00; C07K 1/00

(52) U.S. Cl. 424/176.1; 530/412; 436/547; 530/390.1

(57) ABSTRACT

A method for the sterilization of a virus infected plasma or plasma derivative comprising contacting the plasma or plasma derivative with at least one organic solvents with or without at least one detergent and simultaneously or sequentially with a lipophilic adorbent at a temperature of 30° C. to 70° C.

### BACKGROUND OF THE INVENTION

1. Field of the Invention

The present invention concerns sterilization of virus infected plasma or a plasma derivative (plasma fraction). More particularly, the present invention is directed to rendering plasma or a plasma fraction of suspected high virus infectivity safe for normal fractionation by utilizing solvents, optionally detergents, and a lipophilic absorbent, e.g., a silica.

The present invention also relates to a method for providing immune globulin, such as hepatitis C immune globulin.

2. Background Information

Hyperimmune globulins such as HBIG and RHIG have long been known and are prepared by standard fractionation methods from plasma rich in specific antibodies. Until now, even if the immune globulin was directed against infectious agents such as Hepatitis B, the presence of these antibodies neutralized virus infectivity, and so the starting plasma would not transmit disease either to the workers handling it or to recipients of the hyperimmune globulin preparation derived from it.

However, for certain recently-characterized viruses such as Hepatitis C and HIV, high levels of infectious virus can still be present in plasma containing antibodies to these viruses. Therefore, in order to be able to prepare a hyperimmune globulin specific to these viruses, the antibody-containing plasma must be treated by a virus-inactivation method which can inactivate high levels of virus while preserving the plasma's suitability as a starting material for the fractionation process as well as its content of active antibody.

Numerous attempts have been made to inactivate viruses such as hepatitis viruses in mammalian, especially human, blood plasma. It is the practice in some countries to effect inactivation of the hepatitis B virus in the blood plasma by contacting the plasma with a viral inactivating agent of the type which cross-links with the proteinaceous portion of hepatitis B virus, or which interacts with the nucleic acid of the virus. For instance, it is known to attempt to inactivate hepatitis B virus by contact with an aldehyde such as formaldehyde whereby crosslinking to the protein is effected and the hepatitis B virus is inactivated. It is also known to effect inactivation of the virus by contact with β-propiolactone (BPL), an agent which acts on the nucleic acid of the virus. It is further known to use ultra violet light, especially after a β-propiolactone treatment.

The BPL/UV inactivation procedure discussed above has not so far been adopted in the United States for numerous reasons, one of which lies in the fact that it is thought by some that BPL may render some proteins antigenic. Furthermore, concentrated BPL has been shown to be carcinogenic in animals.

Another method for inactivating viruses in plasma is treatment with the organic solvent tri(n-butyl)phosphate (“TNBP”). TNBP acts by disrupting the virus envelope of lipid-enveloped viruses . TNBP has been shown to inactivate ≧10⁶ infectious doses (ID₅₀) of hepatitis B virus. However, plasma from high risk donors may contain more than 10⁶ ID₅₀ of hepatitis B virus or other viruses, and processing such plasma must still be conducted with great caution to prevent undue exposure of fractionation workers to infectious virus.

Other methods for the inactivation of hepatitis B virus in the plasma are known, but are usually impractical. One method involves the addition of antibodies to the plasma whereby an immune complex is formed. The expense of antibody formation and purification add significantly to the cost of the plasma production; furthermore, there is no assurance that a sufficient quantity of hepatitis B or hepatitis C virus will be inactivated.

U.S. Pat. No. 3,686,395, the entire contents of which are incorporated by reference herein describes the preparation of lipoprotein free, stable and sterile serum by mixing blood serum or plasma with colloidal silica.

A hyperimmune globulin directed against HIV (HIVIG) has been reported (Prince A.M. et al. Proc. Nate. Acad. Sci. (USA) 85:6944-6948, 1988. It was prepared from plasma containing high levels of neutralizing antibodies against HIV which was treated with the organic solvent tri(n-butyl) phosphate prior to fractionation. Neither the treated plasma nor the final product (HIVIG) transmitted HIV infection. However the single virus inactivation method used may not guarantee levels of virus kill high enough to assure workers' safety, especially in the case of hepatitis C (non-A, non-B), which is known to be more difficult to inactivate than HIV. Furthermore it may not result in the degree of virus inactivation (>12 Log₁₀) generally considered necessary for fractionation to be carried out in standard facilities.

### SUMMARY OF THE INVENTION

It is an object of the invention to provide a method for the sterilization of virus infected plasma or plasma fractions.

It is another object of the invention to provide a method of rendering safe plasma or a plasma fractions of suspected high virus infectivity, permitting subsequent fractionation to be conducted in standard facilities.

It is a further object of the invention to provide a method to prepare an immune globulin from virus infected plasma.

It is another object of the present invention to provide a hepatitis C immune globulin.

It is a further object of this invention to provide a method for preparing hepatitis C immune globulin.

It is still another object of the present invention to provide a method for prevention of hepatitis C virus infection after an accidental exposure to the same.

It is further object of the instant invention to provide a method for the treatment and/or prevention of the chronic carrier state of hepatitis C virus.

The above objects, as well as other objects, aims and advantages are satisfied by the present invention.

The present invention concerns a method for the sterilization of a virus (e.g., human immunodeficiency virus, hepatitis C virus or Lassa fever virus) infected plasma or plasma derivative comprising contacting the plasma or plasma derivative with at least one organic solvent with or without at least one detergent and simultaneously or sequentially with one or more lipophilic adsorbents at a temperature of 30 to 70° C. This method serves to render safe plasma or a plasma derivative of suspected high virus infectivity, and permits subsequent fractionation to be carried out in standard facilities.

The present invention also relates to a hepatitis C immune globulin (“HCIG”) prepared from plasma or a plasma derivative infected with hepatitis C virus. Such immune globulin is further characterized as being derived from an anti-HCV(+) plasma pool.

The present invention is also directed to a method for preventing hepatitis C virus infection in a mammalian, e.g., human, patient comprising administering to the patient, e.g., intravenously, an effective therapeutic amount of the hepatitis C immune globulin described above.

The present invention is further directed to a method for the treatment or prevention of the chronic carrier state of hepatitis C virus in a mammalian, e.g., human, patient comprising administering to the patient, e.g., intravenously, an effective therapeutic amount of the hepatitis C immune globulin described above.

The present invention is also directed to a method of rendering a blood product safe from transmission of hepatitis C virus by adding to the blood product a therapeutically effective amount of the hepatitis C immune globulin described above.

The present invention is also directed to a method for preparing an immunoglobulin, e.g., a hepatitis C immune globulin, from a virus infected plasma or plasma derivative comprising contacting the plasma or plasma derivative with one or more organic solvents with or without at least one detergent and simultaneously or sequentially with one or more lipophilic adsorbents at a temperature of 30° C. to 70° C., conducting fractionation and isolating the immune globulin.

The present invention is also directed to a hyperimmune globulin prepared-from a virus infectious plasma, the plasma having been treated to inactivate ≧12 Log₁₀ of virus titer prior to fractionation.

### DETAILED DESCRIPTION OF THE INVENTION

Blood is made up of solids (cells, i.e., erythrocytes, leucocytes, and thrombocytes) and liquid (plasma). The cells contain potentially valuable substances such as hemoglobin, and they can be induced to make other potentially valuable substances such as interferons, growth factors, and other biological response modifiers. The plasma is composed mainly of water, salts, lipids and proteins. The proteins are divided into groups called fibrinogens, serum globulins and serum albumins. Typical antibodies (immune globulins) found in human blood plasma include those directed against infectious hepatitis, influenza, etc.

Immunoglobulins (G, A, M, D and E) are one of the many groups of proteins found in human plasma.

Blood plasma fractionation generally involves the use of organic solvents such as ethanol, ether and polyethylene glycol at low temperatures and at controlled pH values to effect precipitation of a particular fraction containing one or more plasma proteins. The resultant supernatant can itself then be precipitated and so on until the desired degree of fractionation is attained. More recently, separations are based on chromatographic processes have been described. An excellent survey of blood fractionation appears in Kirk-Othmer's Encyclopedia of Chemical Technolocy, Volume 4, pages 25 to 62, the entire contents of which are incorporated by reference herein.

The major components of a cold ethanol fractionation are as follows:

| Fraction | Proteins |
|---|---|
| I | fibrinogen; cold insoluble globulin; |
| | factor VIII; properdin |
| II and III | IgG; IgM; IgA; fibrinogen; beta- |
| | lipoprotein; prothrombin; plasminogen; |
| | plasmin inhibitor; factor V; factor VII; |
| | factor IX; factor X; thrombin; |
| | antithrombin; isoagglutinins; |
| | ceruloplasmin; complement C′1, C′3 |
| IV-1 | alpha₁-lipoprotein, ceruloplasmin, |
| | plasmin-inhibitor; factor IX; peptidase; |
| | alpha-and-beta-globulins |
| IV-4 | transferrin; thyroxine binding globulin; |
| | serum esterase; alpha₁-lipoprotein; |
| | albumin; alkaline phosphatase |
| V | albumin; alpha-globulin |
| VI | alpha₁-acid glycoprotein; albumin |

The above fractionation scheme can serve as a basis for further fractionations. Fraction II and III, for example, can be further fractionated to obtain immune serum globulin (ISG).

One embodiment of the present invention concerns a method for sterilization of a virus infected plasma, e.g., mammalian plasma, e.g., human plasma, or plasma derivative, comprising contacting the plasma or plasma derivative, e.g., serum, with one or more organic solvents, e.g., tri-(n-butyl) phosphate (TNBP) and/or one or more detergents, e.g., “TRITON X-100”, at a temperature of 30° C. to 70° C., preferably 56° C., for ⅙ to 12 hours, preferably for 1 hour. Alternatively, an incubation with solvent and detergents is conducted for 1 to 12 hours at 20 to 50° C., preferably 30° C., followed by a further incubation at 50 to 70° C., preferably 50° C., for ½ to 2 hours. A lipophilic adsorbent is used to remove lipids released by this method. Such method provides a virus kill, for example with respect to human immuodeficiency virus (HIV) of greater than or equal to 14.7 log₁₀.

The lipophilic absorbent is preferably a colloidal silica such as “AEROSIL 380” (Degusa) or a hydrophobic resin, e.g. Cab-O-Sil EH5 (Cabot) or bentonite, agarose, e.g., dodecyl agarose (Sigma), phenyl sepharose or C₆-agarose, and is preferably used in a concentration of 5 to 500 mg/ml.

The present invention can be used to treat plasma or plasma derivatives infected with, for example, one or more of hepatitis B virus (“HBV”), hepatitis C virus (“HCV”), human immunodeficiency virus (“HIV”) or arenaviruses, such as Lassa fever virus.

Preferably the inventive sterilization method is conducted as follows:

(1) contact virus infected plasma or plasma derivative with one or more detergents and organic solvents,

(2) conduct incubation for ⅙ to 12 hours, preferably 4 hours, at 20 to 50° C., preferably 30° C.

(3) raise the temperature to 50° C. to 70° C., preferably 56° C. for ½ to 2 hours, preferably 1 hour,

(4) add the lipophilic adsorbent and

(5) conduct incubation for 1 to 12 hours, preferably 4 hours, at 30 to 50° C., preferably 45° C.

Organic solvents for use in the present invention include a dialkylphosphate or a trialkylphosphate having alkyl groups which contain 1 to 10 carbon atoms, especially 2 to 10 carbon atoms. Illustrative members of trialkylphosphates for use in the present invention include tri(n-butyl) phosphate, tri(t-butyl) phosphate, tri(n-hexyl) phosphate, tri(2-ethylhexyl) phosphate, tri(n-decyl) phosphate, just to name a few. An especially preferred trialkylphosphate is tri(n-butyl) phosphate. Mixtures of different trialkylphosphates can also be employed as well as phosphates having alkyl groups of different alkyl chains, for example, ethyl or di(n-butyl) phosphate. Similarly, the respective dialkylphosphates can be employed, including those of different alkyl group mixtures of dialkylphosphate. Furthermore, mixtures of di- and trialkylphosphates can be employed.

Di- or trialkylphosphates for use in the present invention are employed in an amount between about 0.01 mg/ml and about 100 mg/ml, and preferably between about 0.1 mg/ml and about 10 mg/ml.

Other organic solvents to be used include ethers, with sufficiently high boiling points, e.g., n-propylether, n-butyl ether and tert butyl ethyl ether.

The organic solvent can be used with or without the addition of wetting agents. It is preferred, however, to use a di- or trialkylphosphate in conjunction with a wetting agent. Such wetting agent can be added either before, simultaneously with or after the di- or trialkylphosphate contacts the plasma or plasma derivative. The function of the wetting agent is to enhance the contact of the virus in the plasma or plasma derivative with organic solvent.

Preferred wetting agents are nontoxic detergents. Contemplated nonionic detergents include those which disperse at the prevailing temperature at least 0.1% by weight of the fat in an aqueous solution containing the same when 1 gram detergent per 100 ml of solution is introduced therein. In particular there is contemplated detergents which include polyoxyethylene derivatives of fatty acids, partial esters of sorbitol anhydrides, for example, those products known commercially as “Tween 80”, and “polysorbate 80” and nonionic oil soluble water detergents such as that sold commercially under the trademark “Triton X 100” (oxyethylated alkylphenol). Also contemplated are natural detergents, e.g., sodium oleate and sodium deoxycholate, as well as the “Zwittergents”, which are synthetic zwitterionic detergents known as “sulfobetaines”, such as N-dodecyl-N, N-dimethyl-2-ammonio-1 ethane sulphonate and its congeners or nonionic detergents, such as octyl-beta-D-glucopyranoside.

The detergent can also be used without the addition of an organic solvent. In this case, preferred detergents are non-ionic, e.g., alkylaryl polyether alcohols, or synthetic zwitterionic detergents, e.g., sulfobetaine, or a natural detergent, e.g., sodium oleate or sodium linoleate.

Substances which might enhance the effectiveness of alkylphosphates include reducing agents such as mercaptoethanol, dithiothreitol, dithioerythritol and dithiooctanoic acid. Suitable nonionic surfactants are oxyethylated alkyl phenols, polyoxyethylene sorbitan fatty acid esters, polyoxyethylene acids, polyoxyethylene alcohols, polyoxyethylene oils and polyoxyethylene oxypropylene fatty acids. Some specific examples are the following:

alkylphenoxypolyethoxy (30) ethanol,

polyoxyethylene(2) sorbitan monolaurate,

polyoxyethylene (20) sorbitan monoplamitate,

polyoxyethylene (20) sorbitan monostearate,

polyoxyethylene (20) sorbitan tristerate,

polyoxyethylene (20) sorbitan monooleate,

polyoxyethylene (20) sorbitan trioleate,

polyoxyethylene (20) palmitate,

polyoxyethylene (20) lauryl ether,

polyoxyethylene (20)cetyl ether,

polyoxyethylene (20) stearyl ether,

polyoxyethylene (20) oleyl ether,

polyoxyethylene (25) hydrogenated castor oil and

polyoxyethylene (25) oxypropylene monostearate.

The amount of wetting agent, e.g., detergent, if employed, is not crucial, for example, from about 0.001 wt. % to about 10 wt. %, preferably about 0.01 wt. % to 1.5 wt. %, can be used, wherein the wt. % is based on the total weight of the resultant mixture.

Treatment of the plasma or plasma derivative with the solvent(s), optionally detergent(s) and lipophilic adsorbent is carried out for 1 hour to 12 hours and preferably for 4 hours to 6 hours.

The treatment is normally effective at atmospheric pressure, although subatmospheric and superatmospheric pressures can also be employed.

Normally, after the treatment, the virus inactivating agents (detergents and/or solvents) are removed, although such is not necessary in all instances, depending upon the nature of the virus inactivating agent and the intended further processing of the plasma.

To remove the viral inactivating agent from the plasma, the plasma may be treated as follows:

(1) diafiltration using microporous membranes;

(2) adsorption of desired components on chromatographic or affinity chromatographic supports;

(3) precipitation, e.g., by salting out of the desired components plasma, and

(4) adsorption of the solvent and/or detergent by hydrophobic chromatography.

Removal of lipophilic adsorbents can be carried out by centrifugation or by filtration.

The method of the invention permits the pooling of human blood plasma and the treatment of the pooled human blood plasma in the form of such pooled plasma.

After treatment, the plasma is fractionated either by the Cohn-Oncley Cold-ethanol procedure or by another procedure known to produce a purified immune globuin preparation suitable for human use, in order to provide the desired immune globulin.

The resulting hyperimmune globulin preparation is free of the infectious virus which was present in the starting plasma and is rich in active antibody. It is suitable for all of the uses to which hyperimmune globulin preparations have been applied, some of which are now described for the case of Hepatitis C immune globulin.

a) Prevention of maternal transmission perinatally or to the newborn HCIG is injected intramuscularly or intravenously into the newborn, typically at a dose of from 0.1 to 10 cc/kg (5-500 mg protein) or an amount which can neutralize at least 100 infectious doses of HCV shortly after birth and throughout the post-natal period. Injections are made once every 4-8 weeks and may include use of an HCV vaccine.

b) Post-Exposure Prophylaxis

Individuals exposed to HCV, such as by needlestick injury or during surgery on an HCV-infected individual, are injected at the earliest possible time following exposure with an intravenous injection of 1-10 ml/kg of HCIG (50-500 mg protein), or an amount of anti-HCV which can neutralize at least 100 infectious doses of HCV.

c) Pre-Exposure Prevention

An individual likely to be exposed to HCV because of occupational hazards or because of anticipated travel to an HCV endemic area is injected with HCIG using an amount that can neutralize at least 100 infectious does. Intramuscular injections are preferred, but intravenous injections are acceptable.

d) Prevention of transmission by blood products HCIG is added to red cell or platelet concentrates prior to transfusion or to the receipt of those products, using an amount of HCIG that will neutralize at least 1,000 infectious does of HCV. The addition to a red cell or platelet concentrate typically will be within 24 hours of transfusion, but the HCIG can be added shortly following donation and separation of the blood into its components.

e) HCIG will be injected either intravenously or intramuscularly at monthly intervals into an individual evidenced to already be infected with HCV. A typical dose will be 5-10 cc/kg (250-500 mg protein), or an amount which inactivates at least 1000 infectious doses of HCV. The patient may simultaneously be treated with other anti-viral drugs, e.g. interferon and nucleoside analogs.

In order to more fully illustrate the nature of the invention and the manner of practicing the same, the following non-limiting examples are presented:

### Example 1

### Preparation Of Virus Inactivated Plasma

### Three Step Procedure

The plasma pool was adjusted to 1% TNBP and 1% Triton X 100 in a closed, jacketed, stirrer equipped, vessel. The temperature was raised to 30° C. and stirring continued for 4 hours. The temperature was then raised to 56° C. for 1 hour with continued stirring. Aerosil 380 (Degussa) was then added to 15 gm/liter and stirring was continued for 4 hours at 45° C.

The sterilized plasma was recovered by filtration.

### Example 2

### Preparation Of Virus Inactivated Plasma

### Two Step Procedure

The plasma pool was adjusted to 1% TNBP and 1% Triton X 100 and brought to 56° C. for one hour in a closed stirred vessel. Aerosil 380 was then added to 15 gm/liter and stirring continued for 4 hours at 45° C. The sterilized plasma was recovered by filtration.

### Example 3

### Preparation Of Virus Inactivated Plasma

### Single Step Procedure

The plasma pool was adjusted to 1% TNPB and 1% Triton X 100 in a closed water jacketed, stirrer equipped, vessel containing 15 gm of heat sterilized Aerosil per liter of plasma. The temperature was raised to 56° C. with continued stirring for 1 hour. The sterilized plasma was recovered by filtration.

### Example 4

### Determination Of Veridical Efficacy Of

### Three Step Procedure

In order to determine the extent of inactivation resulting from the individual components of the inventive inactivation method, the following experiment was conducted.

1. The following mixtures were prepared:

| Tube | 1 | 2 | 3 | 4 | 5 |
|---|---|---|---|---|---|
| Plasma⁺ | 1580 μl | 1580 μl | 158 μl | 1580 μl | 1580 μl |
| HIV* | 200 μl | 200 μl | 20 μl | 200 μl | 200 μl |
| TNBP⁺⁺ | 20 μl | — | — | 20 μl | — |
| Triton | 200 μl | — | — | 200 μl | — |
| PBS | — | 200 μl | 22 μl | — | 200 μl |

⁺Pooled Plasma
*Pharmacia (Piscataway, N.J.)/ENI 10,000 fold concentrated HIV virus stock, diluted 1:10
⁺⁺Tri(n-butyl) phosphate
**Triton X 100 diluted 1:10 in PBS (0.1 M NaCl, 0.01 M phosphate, pH 7.2)

2. The above tubes were heated as follows:

Tube

| Temperature | 1 | 2 | 3 | 4 | 5 |
|---|---|---|---|---|---|
| 30° C. | 1 hour | — | — | 4 hours | — |
| 56° C. | 1 hour | — | — | — | 1 hour |

3. 30 mg of Aerosil 380 (Degussa, Interboro, N.J.) were added to tubes 1 and 2 which were then incubated with continuous shaking in a water bath for 4 hours at 45° C.

4. Samples 1 and 2 were transferred to Eppendorf tubes, and centrifuged in a microfuge for 5 minutes at a speed of 14,000 rpm. The supernatants were transferred to new tubes.

5. Samples 1 and 4 were treated with C18 resin (Waters, a division of Millipore Corp., Milford, Mass.) to remove TNBP and Triton X 100 as follows:

a. weigh out 0.24 grams of resin,

b. add 100% isopropanol and allow to settle,

c. wash with 1.5 ml isopropanol, centrifuge and remove supernatant,

d. wash with 3.75 ml of distilled water, centrifuge and remove supernatant,

e. add samples to resin and rock at room temperature for 3 minutes and

f. centrifuge and adsorb supernatants to another batch of resin, wash as described above, centrifuge, sterile filter, and transfer sample to a new tube.

6. Sample 3 was diluted 1:10 with conditioned infection medium (CIM) composed of 10% fetal calf serum in RPMI medium (30% taken from normal CEM cell culture supernatant), 2 μg/ml polybrene.

7. Samples were assayed for infectivity, in quadruplicate, using CEM X174 cells.

The results of the virus titrations, and estimate of virus kill, were as follows:

| | Inactivation | Log₁₀* | Log₁₀ virus |
|---|---|---|---|
| Sample | Steps used | TCID 50/ml | kill** |
| 1 | all | ≦1.0 | ≧5.3 |
| 2 | Aerosil alone | 1.75 | 4.55 |
| 3 | none | 6.3 | — |
| 4 | TNBP/Triton alone | ≦2.0 | ≧4.3 |
| 5 | 56° C., 30 minutes | ≦0.4 | ≧5.9 |
| | alone | | |

*tissue culture infectious doses - 50 (Spearman - Karber method)
**compared to sample No. 3

Summation of the inactivation efficacy of each of the 3 steps used in the combined process (i.e., samples 2, 4 and 5), each of which has an independent mechanism of action, indicates that the combined process has an inactivation process efficacy of ≧14.7 log 10 for HIV.

### Example 5

### Effect Of Inventive Procedure On Antibody Levels

Since Hepatitic C virus cannot be grown in vitro, the only way to measure neutralizing antibody activity against Hepatitis C is in vivo, in chimpanzees. However, because all antibodies are immune globulins, it is possible to assess the effect of the virus inactivation process on antibody activity in a different system, i.e., HIV, and apply the results to Hepatitis C.

1. To each of two aliquots of 17.7 ml filtered normal human plasma add 100 μl of HIVIG (human immunodeficiency virus immune globulin).

2. To tube #1 add 200 μ1 of TNBP, mix and then add 2.0 ml 10% Triton X-100 in PBS. Vortex to mix. To tube #2 add 2.2 ml PBS.

3. Distribute each mixture to two, 15 mL centrifuge tubes.

4. Incubate tubes 1A and 2A 1 hour at 30° C. with shaking with tubes on their sides sealed with parafilm.

5. Incubate tubes 1B and 2B 1 hour at 56° C. as above.

6. Centrifuge all tubes 15 minutes 2000 rmp at room temperature.

7. Vortex tubes and add 150 mg of Aerosil 380 (Degussa). Incubate 4 hours at 45° C. with shaking with tubes on their sides sealed with parafilm.

8. Centrifuge all tubes 15 minutes at 2000 rmp at room temperature.

9. Carefully remove 2 ml of clear plasma from beneath the lipid layers and from above the Aerosil precipitate.

10. Determine anti-HIV ELISA on 2-fold serial dilutions: 1:100, 1:200, 1:400 in duplicate. Plot for ELISA quantitation.

The antibody titer was determined by assuming a constant slope for the curve relating optical density and dilution. The curve for each sample was then fitted by the least squares procedure. The titer was taken as the log₁₀ dilution at the cut-off of the ELISA assay.

| Results | | Log Dilution | 1/Dilution | % |
|---|---|---|---|---|
| Samples | Conditions | Titer | Titer | Recovery¹ |
| 2A | Phosphate Buffered | 2.62 | 417 | 100 |
| | Saline (PBS) + | | | |
| | Aerosil, 30° C. | | | |
| 2B | PBS + Aerosil, | 2.64 | 436 | 104 |
| | 56° C. | | | |
| 1A | TNBP/Triton, + | 2.66 | 457 | 109 |
| | Aerosil 30° C. | | | |
| 1B | TNBP/Triton, + | 2.51 | 323 | 77 |
| | Aerosil 56° C. | | | |

¹Compared to sample 2A

The inventive procedure yields a clear plasma with recovery of antibody activity estimated to be 77%. Application of the inventive procedure to plasma containing antibodies to Hepatitis C will also yield active antibody.

### Example 6

### Fractionation Of Virus Inactivated Plasma

The virus-sterilized plasma containing the discussed active antibody was then fractionated to a purified immune globulin preparation as follows:

Fration II+III was prepared from the starting plasma pool using the Cohn Cold-ethanol fractionation method 6. (E. J. Cohn et.al J. Am. Chem. Soc. 68, 459 )1946).) This was further processed to fraction II (essentially pure immunoglobulin) by the method 9 of Oncley et al. (J. L. Oncley et al J. Am. Chem. Soc. 71, 541 (1949).) The resulting immune globulin was then treated with pepsin at pH4 to render it suitable for intravenous administration and formulated to a 5% solution in an appropriate buffer.

It will be appreciated that the instant specification set forth by way of illustration and not limitation, and that various modifications and changes may be made without departing from the spirit and scope of the present invention.

What is claimed is:

1. A method for sterilization of a virus infected plasma or plasma derivative comprising contacting said plasma or plasma derivative with at least one organic solvent with or without at least one detergent and simultaneously or sequentially with a lipophilic adsorbent at temperatures between 30 to 70° C., such method preserving the suitability of such plasma or plasma derivative for fractionation to an active immune globulin preparation.

2. The method according to claim 1, including the further step of fractionating the plasma or plasma derivative.

3. The method according to claim 1, wherein the lipophilic absorbent has a concentration of 5 to 100 mg/ml and the lipophilic adsorbent is selected from the group consisting of a silicate and a hydrophobic resin.

4. The method according to claim 1, wherein the virus is human immunodeficiency virus.

5. The method according to claim 1, wherein the virus is hepatitis C virus.

6. The method according to claim 1, wherein the virus is Lassa fever virus.

7. The method according to claim 1, wherein the organic solvent is selected from the group consisting of a dialkylphosphate having 1 to 10 carbon atoms a trialkylphosphate having 1 to 10 carbon atoms, an ether with a high boiling point.

8. The method according to claim 7, wherein the organic solvent is a trialkylphosphate and the trialkylphosphate is tri(n-butyl)phosphate.

9. The method according to claim 7, wherein said ether is selected from the group consisting of n-propylether n-butyl ether and tert butyl ethyl ether.

10. The method according to claim 7, wherein the concentration of the organic solvent is 0.01 to 100 mg/ml.

11. The method of claim 7, wherein the concentration of the organic solvent is 0.1 to 10 mg/ml.

12. The method according to claim 1, wherein the virus infected plasma or plasma derivative is first contacted with said at least one organic solvent with or without at least one detergent, then the resultant mixture incubated for 1 to 12 hours at 30 to 70° C., then the lipophilic adsorbent is added, then the resultant mixture is incubated for ½ to 12 hours at 30 to 70° C.

13. The method according to claim 1, wherein the detergent is a nontoxic, nonionic detergent.

14. The method according to claim 1, wherein the detergent is selected from the group consisting of polyoxyethylene derivatives of fatty acids, partial esters of sorbitol anhydrides and nonionic oil soluble water detergents, sodium deoxycholate and synthetic zwitterionic detergents.

15. The method of claim 1, wherein the detergent is used in an amount of 0.001 to 10 wt. %, based on the weight of the total resultant mixture.

16. The method of claim 1, wherein the detergent is used in an amount of 0.01 to 1.5 wt. %, based on the weight of the total resultant mixture.

17. A hepatitis C immune globulin prepared from plasma or a plasma derivative infected with hepatitis C virus.

18. The hepatitis C immune globulin according to claim 17, said immune globulin prepared from an anti-HCV(+) plasma pool.

19. A method for preparing an immune globulin from a virus infected plasma or plasma derivative comprising contacting the plasma or plasma derivative with at least one organic solvent with or without at least one detergent and simutaneously or sequentially with a lipophilic absorbent at a temperature of 30° C. to 70° C., conducting fractionation and isolating the immune globulin.

20. The method according to claim 19, wherein the plasma or plasma derivative is infected with hepatitis C virus and the immune globulin is a hepatitis C immune globulin.

21. The method according to claim 19, wherein the lipophilic adsorbent has a concentration of 5 to 100 mg/ml and the lipophilic adsorbent is selected from the group consisting of a silicate and hydrophobic resin.

22. The method according to claim 19, wherein the organic solvent is selected from the group consisting of dialkylphosphate having 1 to 10 carbon atoms trialkylphosphate having 1 to 10 carbon atoms and another with a high boiling point.

23. The method according to claim 19, wherein the solvent is a trialkylphosphate and the trialkylphosphate is tri(n-butyl)phosphate.

24. The method according to claim 22, wherein the ether is selected from the group consisting of n-propylether, n-butyl ether and tert butyl ethyl ether.

25. The method of claim 19, wherein the detergent is a nontoxic, nonionic detergent.

26. The method according to claim 19, wherein the detergent is selected from the group consisting of polyoxyethlene derivatives of fatty acids, partial esters of sorbitol anhydrides and nonionic oil soluble water detergents, sodium deoxycholate and synthetic zwitterionic detergents.

27. A method for preventing hepatitis C virus infection in a patient after exposure to hepatitis C virus by administering to said patient an effective therapeutic amount of a hepatitis C immune globulin according to claim 17.

28. A method for the treatment or prevention of the chronic carrier state of hepatitis C virus in a patient comprising administering to said patient an effective therapeutic amount of a hepatitis C immune globulin according to claim 17.

29. A method of rendering a blood product safe from transmission of hepatitis C virus by adding to said blood product a therapeutically effective amount of a hepatitis C immune globulin according to claim 17.

30. A hyperimmune globulin prepared from virus infectious plasma, said plasma having been treated to inactivate ≧12 logs of virus prior to a fractionation of said plasma.
